(19) United States
(12) Patent Application Publication
(10) Pub. No.: US 2023/0183653 A1
(43) Pub. Date: Jun. 15, 2023

(54) METHOD FOR PRODUCING STEM CELLS AND METHOD FOR PRODUCING SOMATIC CELLS

(71) Applicants: Koji Tanabe, Palo Alto, CA (US); I Peace, Inc., Palo Alto, CA (US)

(72) Inventors: Koji TANABE, Palo Alto, CA (US); Kenta SUTO, Palo Alto, CA (US)

(73) Assignee: I Peace, Inc., Palo Alto, CA (US)

(21) Appl. No.: 18/064,189

(22) Filed: Dec. 9, 2022

Related U.S. Application Data
(60) Provisional application No. 63/265,267, filed on Dec. 10, 2021.

(51) Int. Cl. C12N 5/074; C12N 15/86

(52) U.S. Cl. CPC C12N 5/0696; C12N 15/86; C12N 2501/23; C12N 2500/42; C12N 2760/18843

(57) ABSTRACT

According to the present disclosure, there is provided a method for producing stem cells including: preparing somatic cells; preparing a chimeric virus including a virus-derived genomic RNA harboring an inducer RNA that induces somatic cells into stem cells and a virus-derived envelope surrounding the genomic RNA, wherein the genomic RNA and the envelope are derived from different viruses; and introducing the inducer RNA into the somatic cells using the chimeric virus.

### BACKGROUND

### Field

The present invention relates to a cell technique and relates to a method for producing stem cells and a method for producing somatic cells.

### Description of Related Art

Induced pluripotent stem (iPS) cells are cells having two characteristic abilities. One is an ability to transform into all the cells that make up the body. The other is to have a semi-permanent proliferative ability. Since iPS cells have these two abilities, they can be applied to a transplantation treatment without rejection by producing iPS cells from their own somatic cells and converting them into target somatic cells. Therefore, iPS cells are considered to be a promising technology for regenerative medicine.

From the invention of iPS cells to the present, many methods of producing iPS cells have been established. Examples of typical methods of producing iPS cells include a method using retroviruses/lentiviruses and a method using an episomal vector.

In the method using retroviruses/lentiviruses, somatic cells are infected with retroviruses or lentiviruses, and genes encoding an initialization factor can be introduced into cells. Retroviruses or lentiviruses allow an initialization factor to be inserted into the genome of somatic cells and induce stable expression of the initialization factor in the cells.

However, the method using retroviruses/lentiviruses has the following problems. First, insertion of the initialization factor into the genome of somatic cells may damage existing genes and promoters, which can cause canceration of the cells. In addition, the initialization factor inserted into the genome may be reactivated after iPS cells are changed to somatic cells. Therefore, cells for transplantation derived from iPS cells have a risk of canceration. For example, in a mouse model, reactivation of the introduced initialization factor is observed in somatic cells, and canceration has been confirmed (for example, refer to Nature 448, 313-317).

An episomal vector is circular DNA and self-amplified in the nucleus. It had been thought that an episomal vector is not integrated into the genome in principle, but in recent studies, it has been reported that fragments of episomal vectors are scattered and inserted into the genome of iPS cells produced in an episomal manner. Therefore, there is a problem of the reprogramming gene remaining in the cells. For example, if c-MYC or KLF4 remains in the cells, it causes canceration. It is expensive to examine whether the reprogramming gene remains in cells. It is not possible to show that all cells in the transplanted cell pool have no insertion of the episomal plasmid into the genome and have no residue.

Since the method using retroviruses/lentiviruses and the method using an episomal vector have the above problems, a method for producing iPS cells using RNA has been proposed (for example, refer to Japanese Patent No. 4478788, Japanese Patent No. 4936482, Japanese Patent No. 5633075, Japanese Patent No. 5963309 and Nature 448, 313-317, Nature Biotechnol 26(3): 313-315, 2008.).

### SUMMARY

There is a need for a method for efficiently introducing factor RNAs into cells. Accordingly, one object of the present invention is to provide a method for producing stem cells and a method for producing somatic cells that can be used to efficiently introduce factor RNAs into cells.

An aspect of the present invention provides a method for producing stem cells including preparing somatic cells; preparing a chimeric virus including a virus-derived genomic RNA harboring an inducer RNA that induces somatic cells into stem cells and a virus-derived envelope surrounding the genomic RNA, wherein the genomic RNA and the envelope are derived from different viruses; and introducing the inducer RNA into the somatic cells using the chimeric virus.

In the method for producing stem cells, the genomic RNA may be derived from paramyxovirus.

In the method for producing stem cells, the genomic RNA may be derived from a Sendai virus.

In the method for producing stem cells, the genomic RNA may include Sendai virus genes in which all functions of M genes, F genes and HN genes among Sendai virus genes including NP genes, P genes/C genes, M genes, F genes, HN genes, and L genes are defected and having a mutation in the L gene that allows permanent gene expression.

In the method for producing stem cells, the envelope may be derived from a measles virus.

In the method for producing stem cells, the somatic cells may be blood cells.

In the method for producing stem cells, the blood cells may be blood cells that do not contain T cells.

In the method for producing stem cells, the blood cells may be T cells.

In the method for producing stem cells, the T cells may be αβT cells.

In the method for producing stem cells, the T cells may be γδT cells.

In the method for producing stem cells, the stem cells may be iPS cells.

The method for producing stem cells may further include applying a phosphate to the somatic cells.

In the method for producing stem cells, the phosphate may be an intermediate or final product of a non-mevalonate pathway.

In the method for producing stem cells, the phosphate may be (E)-4-hydroxy-3-methyl-2-butenyl diphosphate.

The method for producing stem cells may further include applying a bisphosphonate to the somatic cells.

In the method for producing stem cells, the bisphosphonate may be at least one selected from among zoledronic acid, pamidronic acid, alendronic acid, risedronic acid, ibandronic acid, incadronic acid, etidronic acid, minodronic acid, salts thereof, and hydrates thereof.

The method for producing stem cells may further include applying an interleukin to the somatic cells.

In the method for producing stem cells, the interleukin may be at least one selected from the group consisting of IL-2, IL-15, and IL-23.

In the method for producing stem cells, the stem cells may include γδ-TCR rearrangement genes.

In the method for producing stem cells, the stem cells may include αβ-TCR rearrangement genes.

In the method for producing stem cells, when cells into which the inducer RNA is introduced are passaged, at least some of the collected and mixed cells may be seeded.

In the method for producing stem cells, when cells into which the inducer RNA is introduced are passaged, the cells may be seeded at a low concentration.

An aspect of the present invention provides a method for producing somatic cells including preparing stem cells produced according to the method for producing stem cells and inducing somatic cells from the stem cells.

In the method for producing somatic cells, the somatic cells may be blood cells.

In the method for producing somatic cells, the blood cells may be T cells.

In the method for producing somatic cells, when the inducer RNA is introduced into the stem cells, cell masses of the stem cells may be seeded on feeder cells.

In the method for producing somatic cells, the feeder cells may be stromal cells.

According to the present invention, it is possible to provide a method for producing stem cells and a method for producing somatic cells that can be used to efficiently introduce factor RNAs into cells.

### BRIEF DESCRIPTION OF DRAWINGS

FIG. 1 shows images of cells according to Example 1;

FIG. 2 shows images of cells according to Example 1;

FIG. 3 shows images of cells according to Example 1;

FIG. 4 is a dot plot obtained using a flow cytometer according to Example 1;

FIG. 5 shows images of cells according to Example 2;

FIG. 6 is a dot plot obtained using a flow cytometer according to Example 2;

FIG. 7 shows images of cells according to Example 2;

FIG. 8 shows images of cells according to Example 2;

FIG. 9 shows images of cells according to Example 2;

FIG. 10 is a dot plot using a flow cytometer according to Example 3;

FIG. 11 shows dot plots obtained using a flow cytometer according to Example 3;

FIG. 12 shows images of cells according to Example 3;

FIG. 13 shows dot plots obtained using a flow cytometer according to Example 3; and

FIG. 14 shows an image of cells according to Example 4.

### DETAILED DESCRIPTION

Embodiments of the present invention will be described below in detail. Here, the following embodiments are examples for embodying the technical ideas of the invention, and the technical ideas of the invention do not specify the combination of constituent members or the like as in the following. The technical ideas of the invention can be variously modified within the scope of the claims.

A method for producing stem cells according to an embodiment includes preparing somatic cells, preparing a chimeric virus including a virus-derived genomic RNA harboring an inducer RNA that induces somatic cells into stem cells and a virus-derived envelope surrounding the genomic RNA, wherein the genomic RNA and the envelope are derived from different viruses, and introducing the inducer RNA into the somatic cells using the chimeric virus. The stem cells are, for example, induced pluripotent stem cells (iPS cells).

The somatic cells may be derived from humans or derived from non-human animals. Examples of the somatic cells include fibroblasts, blood cells, dental pulp stem cells, keratinocytes, dermal papilla cells, oral epithelial cells, and somatic stem progenitor cells.

The blood cells are isolated from blood. The blood is, for example, peripheral blood or cord blood, but is not limited thereto. The blood may be collected from an adult or a minor. During blood sampling, an anticoagulant such as ethylene-diamine-tetraacetic acid (EDTA), heparin, and a biological preparation standard blood preservative solution A (ACD-A) may be used.

The blood cells include, for example, nucleated cells such as monocytes, neutrophils, eosinophils, basophils, and lymphocytes, and do not include red blood cells, granulocytes, and platelets. The blood cells may be, for example, vascular endothelial progenitor cells, blood stem/progenitor cells, T cells, or B cells. T cells may be, for example, αβT cells or γδT cells. The blood cells need not be γδT cells. The blood cells may be blood cells that do not contain T cells.

When the somatic cells are the blood cells, optionally, a phosphate may be applied to the blood cells. The phosphate may be an intermediate or final product of a non-mevalonate pathway. The non-mevalonate pathway is a synthetic pathway for isopentenyl pyrophosphate (IPP) that does not pass through mevalonic acid. Examples of intermediates of the non-mevalonate pathway include 1-deoxy-D-xylulose 5-phosphate (DOXP), 2-C-methyl-D-erythritol 4-phosphate (MEP), 4-diphosphocytidyl-2-C-methylerythritol (CDP-ME), 4-diphosphocytidyl-2-C-methyl-D-erythritol-2-phosphate (CDP-MEP), 2-C-methyl-D-erythritol-2,4-cyclopyrophosphate (MEcPP), and (E)-4-hydroxy-3-methyl-2-butenyl diphosphate (HMB-PP).

When the phosphate is applied to the blood cells, the phosphate may be added to a medium in which the blood cells are cultured. The concentration of the phosphate in the medium is, for example, 1 μmol/L or more and 50 μmol/L or less, 3 μmol/L or more and 20 μmol/L or less, or 5 μmol/L or more and 12 μmol/L or less. The blood cells may be cultured in a medium to which the phosphate is added for 1 day or more, 2 days or more, or 3 days or more. The blood cells may be cultured in a medium to which a phosphate is added for 14 days or less, and 10 days or more, or 7 days or more.

When the somatic cells are the blood cells, optionally, a bisphosphonate may be applied to the blood cells. Examples of bisphosphonates include zoledronic acid, pamidronic acid, alendronic acid, risedronic acid, ibandronic acid, incadronic acid, etidronic acid, minodronic acid, salts thereof, and hydrates thereof.

When the bisphosphonate is applied to the blood cells, the bisphosphonate may be added to a medium in which the blood cells are cultured. The concentration of the bisphosphonate in the medium is, for example, 1 μmol/L or more and 50 μmol/L or less, 3 μmol/L or more and 20 μmol/L or less, or 5 μmol/L or more and 12 μmol/L or less. The blood cells may be cultured in a medium to which the bisphosphonate is added for 1 day or more, 2 days or more, or 3 days or more. The blood cells may be cultured in a medium to which the bisphosphonate is added for 14 days or less, 10 days or more, or 7 days or more.

When the somatic cells are the blood cells, optionally, an interleukin may be applied to the blood cells. Examples of interleukins include IL-2, IL-15, and IL-23.

When the interleukin is applied to the blood cells, the interleukin may be added to a medium in which the blood cells are cultured. The concentration of the interleukin in the medium is, for example, 5 ng/mL or more and 100 ng/mL or less, 10 ng/mL or more and 90 ng/mL or less, or 15 ng/mL or more and 80 ng/mL or less. The blood cells may be cultured in a medium to which the interleukin is added for 1 day or more, 2 days or more, or 3 days or more. The blood cells may be cultured in a medium to which the interleukin is added for 14 days or less, 10 days or more, or 7 days or more.

The interleukin may be applied after a phosphate and/or bisphosphonate is applied to the blood cells. The phosphate and/or bisphosphonate and the interleukin may be applied to the blood cells at the same time. The phosphate and/or bisphosphonate may be applied after the interleukin is applied to blood cells.

Examples of the mediums in which the somatic cells are cultured include RPMI1640 mediums, minimum essential mediums (α-MEM), Dulbecco's modified eagle mediums (DMEM), F12 mediums, mediums containing no animal components (RPMI medium), and mediums containing no FBS, but the present invention is not limited thereto. The period for culturing the somatic cells before the inducer is introduced is arbitrary, but may be, for example, 2 days or less.

Next, the inducer is introduced into the somatic cells, and the stem cells are induced from the somatic cells. In inducing stem cells, induction refers to reprogramming, initialization, transformation, cell fate change (cell fate reprogramming) or the like.

The inducer introduced into the somatic cells is RNA. RNA may be mRNA. Examples of the inducers include OCT3/4, SOX2, KLF4, and c-MYC. As the inducer, M30 which is improved OCT3/4 may be used. In addition, the inducer may be at least one selected from the group consisting of LIN28A, FOXH1, LIN28B, GLIS1, p53-dominant negative, p53-P275S, L-MYC, NANOG, DPPA2, DPPA4, DPPA5, ZIC3, BCL-2, E-RAS, TPT1, SALL2, NAC1, DAX1, TERT, ZNF206, FOXD3, REX1, UTF1, KLF2, KLF5, ESRRB, miR-291-3p, miR-294, miR-295, NR5A1, NR5A2, TBX3, MBD3sh, TH2A, TH2B, and P53DD. These RNAs for inducers are available from TriLink. Here, although the gene symbols are described here as those of humans, this is not intended to limit the species by uppercase or lowercase letters. For example, denoting in all uppercase letters does not exclude inclusion of mouse or rat genes. However, in the examples, the gene symbols are shown according to the species actually used.

The inducer RNA may be modified with at least one selected from the group consisting of pseudouridine (Ψ), 5-methyluridine (5meU), N1-methylpseudouridine (me1Ψ), 5-methoxyuridine (5moU), 5-hydroxymethyluridine (5hmU), 5-formyluridine (5fU), 5-carboxymethyl ester uridine (5camU), thioguanosine (thG), N4-methylcytidine (me4C), 5 methylcytidine (m5C), 5-methoxycytidine (5moC), 5-hydroxymethylcytidine (5hmC), 5-hydroxycytidine (5hoC), 5-formylcytidine (5fC), 5-carboxylcytidine (5caC), N6-methyl-2-aminoadenosine (m6DAP), diaminopurine (DAP), 5 methyluridine (m5U), 2′-O-methyluridine (Um or m2′-OU), 2-thiouridine (s2U), and N6-methyl adenosine (m6A).

The inducer RNA may be polyadenylated.

The inducer RNA may be prepared by polyadenylation of (IVT) RNA transcribed in vitro. RNAs may be polyadenylated during IVT, using a DNA template that encodes poly (A) ends. RNA may be capped. In order to maximize the efficiency of expression in cells, it is preferable that most RNA molecules contain a cap. RNA may have a 5′ cap [m7G(5′)ppp(5′)G] structure. This sequence is a sequence that stabilizes RNA and promotes transcription. 5′ triphosphate may be removed from RNA having 5′ triphosphate according to a dephosphorylation treatment. RNA may have [3′O-Me-m7G(5′)ppp(5′)G] as Anti-Reverse Cap Analog (ARCA). ARCA is a sequence that is inserted before a transcription start point, doubling the transcription efficiency of RNA. RNA may have a PolyA tail.

In addition, the inducer RNA may be a replicative RNA having a self-proliferation ability. Replicative RNA is RNA having a self-proliferation ability, and unlike normal RNA, it also has an ability to express proteins necessary for RNA replication. The replicative RNA is derived from the Venezuelan equine encephalitis (VEE) virus, which is a type of alphavirus. When replicative RNA is transfected into cells, since RNA that continues to produce an inducer can be expressed in cells, it is possible to omit introduction of cells into RNA a plurality of times.

The replicative RNA sequence may include a sequence obtained from an alphavirus selected from the group consisting of alphavirus replicon RNA, eastern equine encephalitis virus (EEE), Venezuelan equine encephalitis virus (VEE), Everglades virus, Mucambo virus, Pixuna virus, and western equine encephalitis virus (WEE).

In addition, the replicative RNA may include sequences obtained from an alphavirus selected from the group consisting of Sindbis virus, Semliki Forest virus, Middelburg virus, Chikungunya virus, O'nyong-nyong virus, Ross River virus, Barmah Forest virus, Getah virus, Sagiyama virus, Bebaru virus, Mayaro virus, Una virus, Aura virus, Whataroa virus, Babanki virus, Kyzylagach virus, Highlands J virus, Fort Morgan virus, Ndumu virus, and Buggy Creek virus.

Replicative RNA contains, for example, from 5′ to 3′, (VEE RNA replicase)-(promoter)-(RF1)-(self-cleaving peptide)-(RF2)-(self-cleaving peptide)-(RF3)-(IRES or a core promoter)-(RF4)-(IRES or any promoter)-(optionally selectable marker)-(VEE 3′UTR and poly A tail)-(optionally selectable marker)-promoter. The above RF1-4 is a factor that induce dedifferentiate of somatic cells into pluripotent cells. The above RF2-3, RF3-4, and RF4 are optional. The above RF1-4 may be selected from the group consisting of OCT3/4, KLF4, SOX-2, c-MYC, LIN28A, LIN28B, GLIS1, FOXH1, p53-dominant negative, p53-P275S, L-MYC, NANOG, DPPA2, DPPA4, DPPA5, ZIC3, BCL-2, E-RAS, TPT1, SALL2, NAC1, DAX1, TERT, ZNF206, FOXD3, REX1, UTF1, KLF2, KLF5, ESRRB, miR-291-3p, miR-294, miR-295, NR5A1, NR5A2, TBX3, MBD3sh, TH2A, and TH2B.

The inducer is introduced into the somatic cells using the chimeric virus including the virus-derived genomic RNA harboring the inducer RNA and the virus-derived envelope surrounding the genomic RNA, wherein the genomic RNA and the envelope are derived from different viruses.

The chimeric virus genomic RNA may be derived from paramyxoviruses. The chimeric virus genomic RNA may be derived from Sendai viruses. The Sendai viruses are viruses whose genome is RNA and the Paramyxoviridae family belonging to the order Mononegavirales. A wild-type Sendai virus has an RNA genome and an envelope composed of a lipid double membrane encapsulating RNA.

The chimeric virus genomic RNA may be a stealth RNA vector.

The chimeric virus genomic RNA may include Sendai virus genes in which all functions of M genes, F genes and HN genes among Sendai virus genes including nucleocapsid protein (NP) genes, phosphorylated protein (P) genes/C protein (C) genes, matrix protein (M) genes, membrane fusion protein (F) genes, hemagglutinin-neuraminidase (HN) genes, and large protein (L) genes are defected and having a mutation in the L gene that allows permanent gene expression, and an inducer RNA.

NP genes, P genes/C genes, and L genes are involved in transcription and replication of virus vectors. F genes, M genes, and NH genes are involved in virus particle formation. Therefore, virus vectors in which all functions of F genes, M genes, and NH genes are defected cannot form new virus particles after infecting cells. F genes, M genes, and NH genes may be defected in the genomic RNA in order to eliminate all functions of F genes, M genes, and NH genes.

The gene L, which has the mutation that allows permanent gene expression, encodes a large protein with valine at position 1618 in the amino acid sequence. The virus vector having the mutated L gene has a reduced ability to induce interferon, and thus lacks cytotoxicity and has persistent infectivity. Therefore, expression of the loaded inducer RNA is sustained in the cells.

After the stem cells are induced from the somatic cells, in order to remove the virus vector from the cells, siRNA targeting at least any of NP genes, P genes, and mutated L genes may be introduced into the cells. For example, siRNA targeting a region including a nucleotide at position 527 or 1913 in the mutated L gene may be introduced into the cells.

Alternatively, a target sequence of undifferentiated cell-specific microRNA may be added to a non-coding region of at least any of NP genes, P genes, and mutated L genes. Examples of undifferentiated cell-specific microRNA include miR-302a. When the stem cells are induced from the somatic cells, expression of undifferentiated cell-specific miRNA such as miR-302a is induced in the cells. When undifferentiated cell-specific miRNA binds to the target sequence, expression of at least any of NP genes, P genes, and mutated L genes is inhibited, and the virus vector is removed from the cells. For example, a target sequence of miR-302a is added to the mutated L gene.

The chimeric virus genomic RNA may have, from the 3′ end side, NP genes, P genes, and C genes of Sendai viruses and mutated L genes that allow permanent gene expression. The genomic RNA may have the inducer RNA between the C gene and the mutated L gene. The genomic RNA may have a fluorescent protein RNA between the C gene and the mutated L gene. Examples of fluorescent proteins include enhanced green fluorescent proteins (EGFP). The genome may have a drug-resistant gene RNA between the C gene and the mutated L gene.

The chimeric virus envelope is derived from, for example, measles virus.

TOKIWA Bio Inc produced a chimeric virus including a genomic RNA having, from the 3′ end side, NP genes, P genes, and C genes of Sendai viruses and mutated L genes and RNA of EGFP and an inducer RNA between the C gene and the mutated L gene, and a measles virus-derived envelope surrounding the genomic RNA as MSRV-1 based on our request. TOKIWA Bio Inc produced a chimeric virus in which a target sequence of miR-302a was added to the MSRV-1 mutated L gene as MSRV-2 based on our request.

As an indicator of chimeric virus titer, the multiplicity of infection (MOI) is exemplified. The MOI of chimeric virus is, for example, 0.1 to 100.0 or 1.0 to 50.0.

The inducer may be introduced into the somatic cells that are adherently cultured or the inducer may be introduced into the somatic cells that are suspend-cultured in a gel medium.

The somatic cells into which the inducer has been introduced may be cultured using a feeder free basement membrane matrix such as Matrigel (Corning), CELLstart (registered trademark, ThermoFisher), or Laminin511 (iMatrix-511, nippi).

As a medium for culturing the somatic cells into which the inducer has been introduced, a stem cell medium such as a human ES/iPS medium, for example, Stemfit (Ajinomoto), can be used.

However, the stem cell medium is not limited thereto, and various stem cell media can be used. For example, Primate ES Cell Medium, mTeSR1, TeSR2 (STEMCELL Technologies) and the like may be used. The stem cell medium is put into, for example, a dish, a well, or a tube. After the inducer is introduced into the somatic cells, the cells may be initialized while being two-dimensionally cultured or the cells may be initialized while being suspend-cultured, three-dimensionally cultured, or stir-cultured. The expanding and culturing may be two-dimensional culturing, suspend-culturing, three-dimensional culturing, or stir-culturing.

After the inducer is introduced into the somatic cells and the cells are cultured, the cells to which the inducer has been introduced are collected, and at least some of the collected and mixed cells are seeded and passaged in a medium, which is performed at least once. In passaging, clones of the cells into which the inducer has been introduced may be mixed. In passaging, different clones of the cells into which the inducer has been introduced may be mixed together. Then, cells into which the inducer has been introduced are collected, and at least some of the collected and mixed cells are seeded and passaged in a medium, which may be performed a plurality of times. Until stem cells are derived, cells into which the inducer has been introduced may be collected, and at least some of the collected and mixed cells may be seeded and passaged in a medium. It should be noted that all of the collected and mixed cells may be seeded in a medium.

Here, collecting the cells into which the inducer has been introduced and seeding and passaging at least some of the collected and mixed cells in the medium refers to, for example, passaging the cells into which the inducer has been introduced without distinguishing them according to their gene expression state. For example, during passaging, the cells into which the inducer has been introduced may be seeded in the same culture container without distinguishing them according to their gene expression state. Alternatively, collecting the cells into which the inducer has been introduced and seeding and passaging at least some of the collected and mixed cells in the medium refers to, for example, passaging the cells into which the inducer has been introduced without distinguishing them according to their degree of reprogramming. For example, during passaging, the cells into which the inducer has been introduced may be seeded in the same culture container without distinguishing them according to their degree of reprogramming.

Alternatively, collecting the cells into which the inducer has been introduced and seeding and passaging at least some of the collected and mixed cells in the medium refers to, for example, passaging the cells into which the inducer has been introduced without distinguishing them according to their form. For example, during passaging, the cells into which the inducer has been introduced may be seeded in the same culture container without distinguishing them according to their form. Alternatively, collecting the cells into which the inducer has been introduced and seeding and passaging at least some of the collected and mixed cells in the medium refers to, for example, passaging the cells into which the inducer has been introduced without distinguishing them according to their size. For example, during passaging, the cells into which the inducer has been introduced may be seeded in the same culture container without distinguishing them according to their size.

Alternatively, collecting the cells into which the inducer has been introduced and seeding and passaging at least some of the collected and mixed cells in the medium refers to passaging the cells into which the inducer has been introduced without cloning. For example, when passaging is performed without cloning, it is not necessary to pick up colonies formed by the cells into which the inducer has been introduced. For example, when passaging is performed without cloning, a plurality of colonies formed by the cells into which the inducer has been introduced may not be isolated from each other. For example, during passaging, cells forming a plurality of different colonies may be mixed and seeded in the same culture container. In addition, for example, when passaging is performed without cloning, it is not necessary to clone a single colony formed by the cells into which the inducer has been introduced. For example, during passaging, colonies may be mixed and seeded in the same culture container.

For example, in the case where the cells into which the inducer has been introduced are adherently cultured, the adherently cultured cells may be collected, and at least some of the collected and mixed cells may be seeded and passaged in a medium. For example, during passaging, cells may be detached from the culture container and at least some of the detached and mixed cells may be seeded in the same culture container. For example, cells may be detached from the culture container with a dissociation solution and all detached and mixed cells may be passaged. For example, cells that do not form colonies may be passaged. In the case where the cells into which the inducer has been introduced are suspend-cultured, all suspend-cultured cells may be passaged.

When the cells into which the inducer has been introduced are passaged, the cells may be seeded in a medium or culture container at a low concentration. Here, the low concentration is, for example, 1 cell/cm² or more, 0.25×10⁴ cells/cm² or less, 1.25×10³ cells/cm² or less, 0.25×10³ cells/cm² or less, 0.25×10² cells/cm² or less, or 0.25×10¹ cells/cm² or less. Alternatively, the low concentration is a concentration at which 10 cells or less, 9 cells or less, 8 cells or less, 7 cells or less, 6 cells or less, 5 cells or less, 4 cells or less, 3 cells or less, or 2 cells or less can come into contact with each other, and 11 or more cells do not come into contact with each other. Here, there may be a plurality of cell masses in which 10 or less cells come into contact with each other. Alternatively, the state in which the entire bottom surface of the cell container is covered with cells is regarded as 100% confluence, and the low concentration is 5% or less confluence, 4% or less confluence, 3% or less confluence, 2% or less confluence, 1% or less confluence, 0.5% or less confluence, 0.1% or less confluence, 0.05% or less confluence, or 0.01% or less confluence. Alternatively, the low concentration is, for example, a concentration at which single cells do not come into contact with each other in the seeded cells. For example, single cells may be seeded in wells of a well plate. The well plate may be a 12-well plate or a 96-well plate. According to the findings by the inventors, when the cells into which the inducer has been introduced are passaged, the cells are seeded in a medium at a low concentration, and thus the residual chimeric virus in the stem cells induced from the cells can be minimized. In the induced stem cells, the proportion of cells in which the chimeric virus remains is, for example, 4% or less, 3% or less, 2% or less, 1% or less, 0.5% or less or 0%.

The cells into which the inducer has been introduced may be cultured and passaged in a closed culture container. In the closed culture container, for example, gases, viruses, microorganisms and impurities are not exchanged with the outside. In addition, the cells into which the inducer has been introduced may be expanded and cultured in two-dimensional culture or may be expanded and cultured in three-dimensional culture.

After the cells into which the inducer has been introduced are induced to stem cells and stem cells are established, all adherently cultured cells may be cryopreserved as stem cells. For example, all cells detached from the culture container with a dissociation solution may be cryopreserved as stem cells. In addition, after the cells into which the inducer has been introduced are induced to stem cells, all suspend-cultured cells may be cryopreserved as stem cells.

The induced stem cells can express undifferentiated cell markers Nanog, OCT4, SOX2 and the like. The induced stem cells can express TERT. The induced stem cells can exhibit telomerase activity.

In addition, it is possible to determine as to whether the stem cells are induced from the somatic cells, for example, based on the morphology of the cells. For example, the induced stem cells can form flat colonies similar to ES cells and express alkaline phosphatase. Alternatively, determination of whether the stem cells are induced from the somatic cells may be performed by analyzing whether at least one surface marker selected from among cell surface markers TRA-1-60, TRA-1-81, SSEA-1, and SSEA5 which indicate undifferentiation, with a flow cytometer, is positive. TRA-1-60 is an iPS/ES cell-specific antigen, and is not detected in somatic cells. Since iPS cells can be produced only from TRA-1-60 positive fractions, TRA-1-60 positive cells are considered to be iPS cells.

The induced stem cells may include, for example, a γδ-TCR rearrangement gene. The γδ-TCR rearrangement gene is a gene encoding TCR in which the TCRγ region and the TCRδ region have been rearranged. The TCRγ region includes Vγ-Jγ. The TCRδ region includes Vδ-Dδ-Jδ. The induced stem cells include, for example, γδ-TCR rearrangement genes including J1/J2 genes. In addition, the induced stem cells may include, for example, an αβ-TCR rearrangement gene.

A method for producing somatic cells according to an embodiment includes preparing the stem cells produced according to the method for producing the stem cells and inducing the somatic cells from the stem cells.

The source of stem cells is arbitrary. The stem cells may be derived from αβT cells. Examples of the somatic cells include those described above. The somatic cells may be blood cells. The blood cells may be αβT cells. The somatic cells from which the stem cells are derived and the somatic cells induced from the stem cells may be of the same type. The method for inducing the blood cells from the stem cells is not particularly limited. For example, the prepared cells are cultured in a medium containing GSK3 inhibitors such as CHIR99021, bone morphogenetic proteins such as BMP-4, and growth factors such as VEGF for 4 days. In addition, the cells are cultured in a medium containing ALK5 inhibitors such as SB431542, growth factors such as VEGF and bFGF, and a stem cell factor (SCF) for 2 days. In addition, the cells are cultured in a medium containing growth factors such as VEGF, SCF, interleukins such as IL-3 and IL-6, cytokine such as Flt3L, and erythropoietin (EPO) for 2 days. In addition, the cells are cultured in a medium containing SCF, interleukins such as IL-6, and EPO. Thereby, the blood cells are induced from the stem cells.

Alternatively, the stem cells may be seeded on stromal cells and the blood cells may be induced from the stem cells. The stromal cells may be derived from bone marrow. The stromal cells may be OP9 cells. The OP9 cells do not produce a macrophage colony-stimulating factor (M-CSF) and have a function of supporting differentiation of the stem cells into the blood cells. For example, stem cell colonies are divided into a plurality of cell masses, and the cell masses of the stem cells are seeded on the OP9 cells as feeder cells. Interleukin-7 and a stem cell factor may be added to the medium. Thereby, the blood cells are induced from the stem cells. For example, the induced blood cells are positive for CD34 and CD43.

CD4-positive and CD8-positive cells may be selected from CD34-positive and CD43-positive cells. The CD4-positive and CD8-positive cells may be cultured in a medium containing anti-CD3 antibodies, anti-CD28 antibodies, and interleukin-2, and induce CD8 mono-positive cells, which are CD4-negative.

### Example 1

A blood cell medium (RPMI Gibco) containing a stem cell factor having a final concentration of 50 ng/mL (SCF, Peprotech), a granulocyte-macrophage colony-stimulating factor having a final concentration of 10 ng/mL (GM-CSF, Peprotech) and interleukin-3 having a final concentration of 20 ng/mL (IL-3, Peprotech) was prepared.

MSRV-1 and MSRV-2 (TOKIWA Bio Inc.) were prepared as chimeric viruses harboring hOCT3/4, hKLF4, hSOX2, and h-c-MYC.

SRV-2 (TOKIWA Bio Inc.) which was a virus including a genomic RNA including, from the 3′ end side, NP genes, P genes, C genes, and mutated L genes of Sendai viruses and RNA of EGFP and an inducer RNA between the C gene and the mutated L gene, and a Sendai virus-derived envelope surrounding the genomic RNA was prepared. In SRV-2, a target sequence of miR-302a was added to the mutated L gene. SRV-2 harbored hOCT3/4, hKLF4, hSOX2, and h-c-MYC.

10 mL of the blood cell medium was put into a 15 mL tube and human blood cells were additionally added. Next, 200 g of the blood cells was centrifuged for 5 minutes. After centrifugation, the blood cells were collected, the number of the blood cells was counted, and 2×10⁵ blood cells were isolated. 10 mL of the blood cell medium was put into a 15 mL tube, and 2×10⁵ blood cells were additionally added. Next, 200 g of the blood cells was centrifuged for 5 minutes. After centrifugation, the blood cell medium was removed from the tube, and 1 mL of the new blood cell medium was put into the tube. The blood cells were suspended in the blood cell medium, and the blood cells were seeded on a laminin-coated dish. Then, the blood cells were cultured in an incubator at 37° C. for 6 days and non-T cells were proliferated. Here, the medium was added to the dish on the 3^{rd} day from the initiation of the culture.

The dish was taken out from the incubator, the blood cell medium in the dish was removed, and 1 mL of the new blood cell medium was added to the dish. In addition, MSRV-1, MSRV-2, or SRV-2 was added to the blood cell medium in each dish. Thereby, KLF4, OCT3/4, SOX2, and c-MYC were introduced into the blood cells. The multiplicity of infection (MOI) was adjusted from 20 to 30.

On the 2^{nd} day after the virus was added to the blood cell medium, the blood cell medium was detached from the dish, the cells were washed with a stem cell medium (StemFit, registered trademark, Ajinomoto), and 1 mL of the stem cell medium was then added to the dish. Then, the stem cell medium was replaced every day. In addition, whenever colonies were formed, passaging was performed.

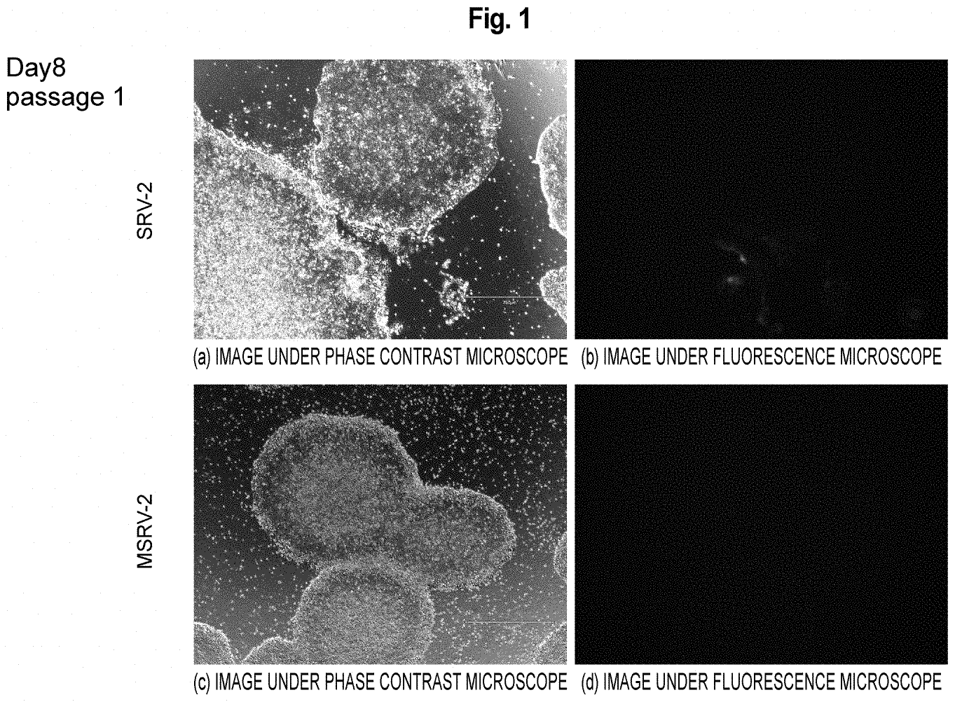

FIG. 1 shows images of iPS-cell-like cells after passaging was performed once on the 8th day. Fluorescence was observed in the cells into which SRV-2 was introduced. This indicates that the virus vector remained in the cells. No fluorescence was observed in the cells into which MSRV-2 was introduced. This indicates that no chimeric virus vector remained in the cells.

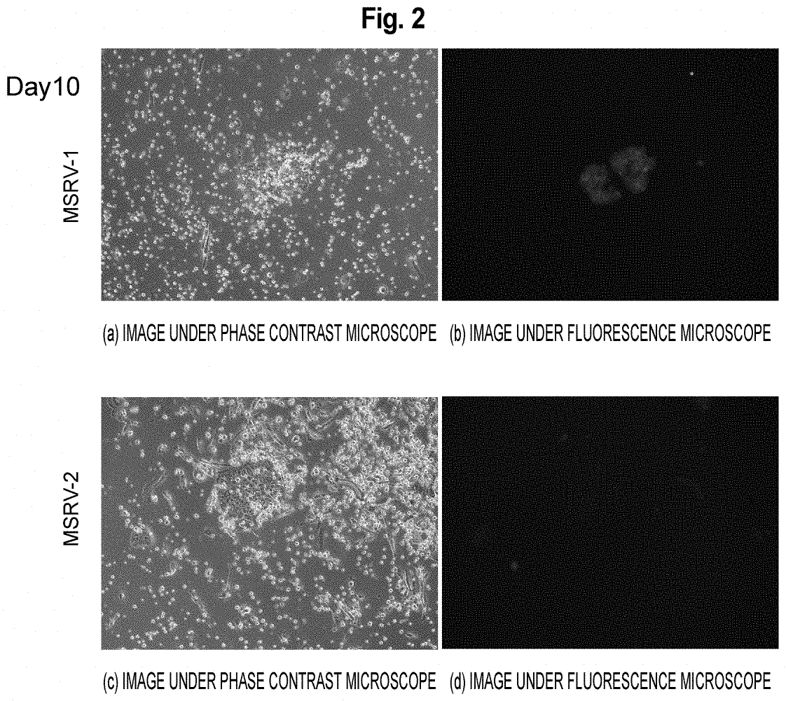

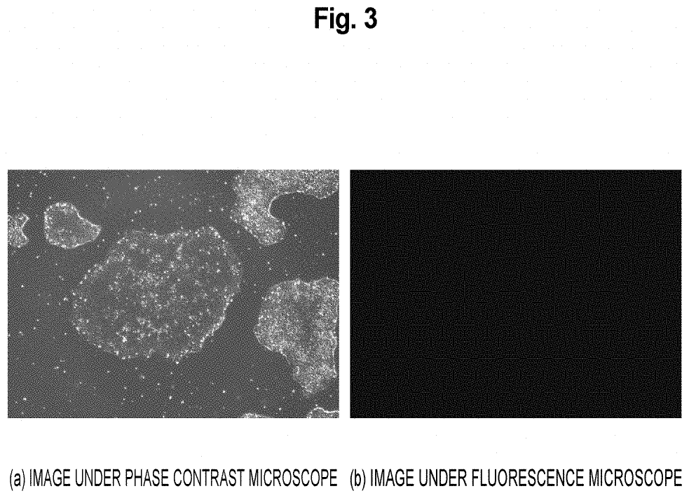

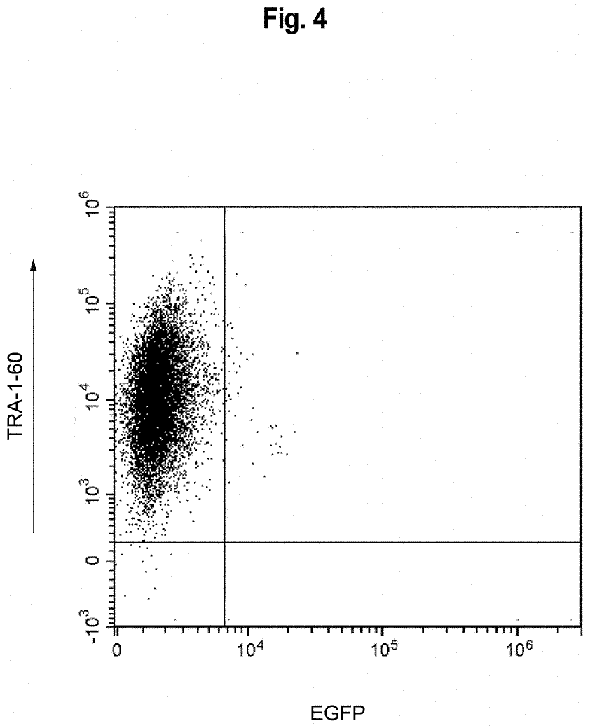

FIG. 2 shows images of the cells on the 10th day. On the 15th day after passaging three times, as shown in FIG. 3, no fluorescence was observed in the cells into which MSRV-2 was introduced. In addition, the cells into which MSRV-2 was introduced were analyzed with a flow cytometer, as shown in FIG. 4. They were EGFP negative and TRA-1-60 positive. Example 1 shows that the chimeric virus genomic RNA disappeared from cells faster than the non-chimeric virus genomic RNA.

### Example 2

A blood cell medium (RPMI Gibco) containing interleukin-2 having a final concentration of 20 ng/mL (IL-2, Peprotech), GlutaMAX supplement having a final concentration of 1% (registered trademark, ThermoFisher), a fetal bovine serum having a final concentration of 10% (FBS, cytiva), and Dynabeads Human T-Activator CD3/CD28 having a final concentration of 12.5 μL/mL (ThermoFisher) was prepared.

MSRV-1, MSRV-2, and SRV-2 (TOKIWA Bio Inc.) were prepared.

SRV-1 (TOKIWA Bio Inc.) which was a virus including a genomic RNA including, from the 3′ end side, NP genes, P genes, C genes and mutated L genes of Sendai viruses, and RNA of EGFP and an inducer RNA between the C gene and the mutated L gene, and a Sendai virus-derived envelope surrounding the genomic RNA was prepared. In SRV-1, a target sequence of miR-302a was not added to the mutated L gene. SRV-1 harbored hOCT3/4, hKLF4, hSOX2, and h-c-MYC.

10 mL of the blood cell medium was put into a 15 mL tube, and human αβT cells were additionally added. Next, 200 g of the αβT cells was centrifuged for 5 minutes. After centrifugation, the αβT cells were collected, the number of the αβT cells was counted, and 5×10⁵ αβT cells were isolated. 10 mL of the blood cell medium was put into a 15 mL tube, and 5×10⁵ αβT cells were additionally added. Next, 200 g of the αβT cells was centrifuged for 5 minutes. After centrifugation, the blood cell medium was removed from the tube, and 1 mL of the new blood cell medium was put into the tube. The αβT cells were suspended in the blood cell medium, and the αβT cells were seeded in a non-laminin-coated 12-well dish. Then, the αβT cells were cultured in an incubator at 37° C. for 2 days.

5×10⁴ αβT cells were isolated, and the αβT cells were seeded in a 6-well dish containing the blood cell medium. In addition, MSRV-1, MSRV-2, SRV-1, or SRV-2 was added to the blood cell medium of each dish. Thereby, KLF4, OCT3/4, SOX2, and c-MYC were introduced into the αβT cells. The multiplicity of infection (MOI) was adjusted from 10 to 30.

On the 3^{rd} day after the virus was added to the blood cell medium, the blood cell medium was removed from the dish, the cells were washed with a stem cell medium (StemFit, registered trademark, Ajinomoto), and 1 mL of the stem cell medium was then added to the dish. Then, the medium was replaced once every two days, and passaging was performed whenever colonies were formed.

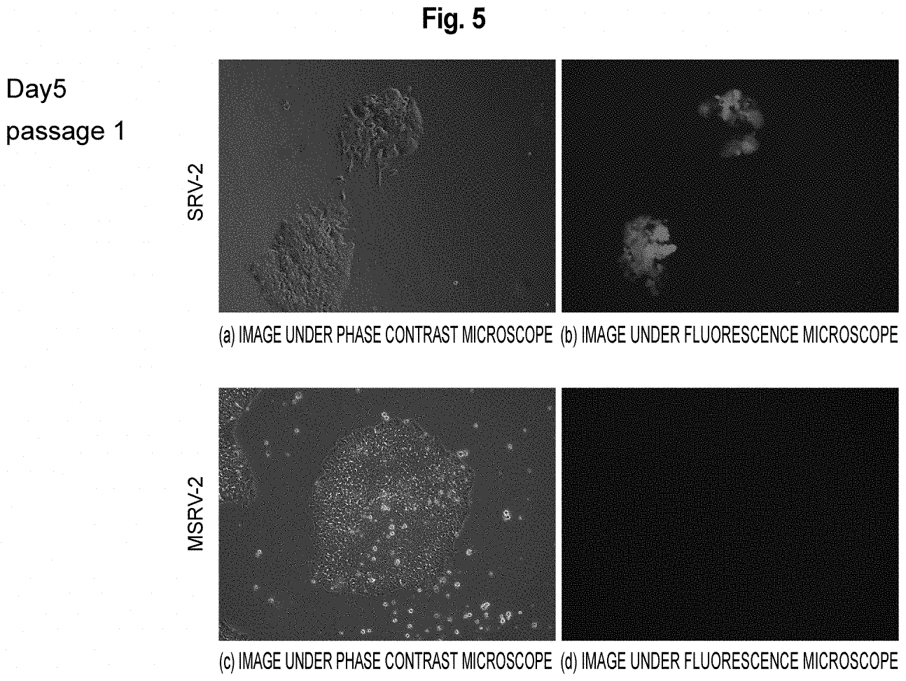

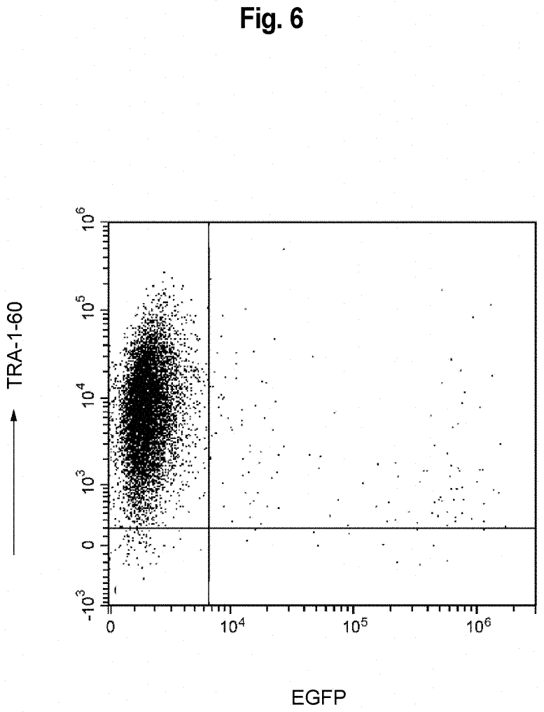

FIG. 5 shows images of iPS-cell-like cells after passaging was performed once on the 5th day. Fluorescence was observed in the cells into which SRV-2 was introduced. This indicates that the virus vector remained in the cells. No fluorescence was observed in the cells into which MSRV-2 was introduced. This indicates that no chimeric virus vector remained in the cells. In addition, the cells into which MSRV-2 was introduced were analyzed with a flow cytometer, as shown in FIG. 6. They were EGFP-negative and TRA-1-60-positive.

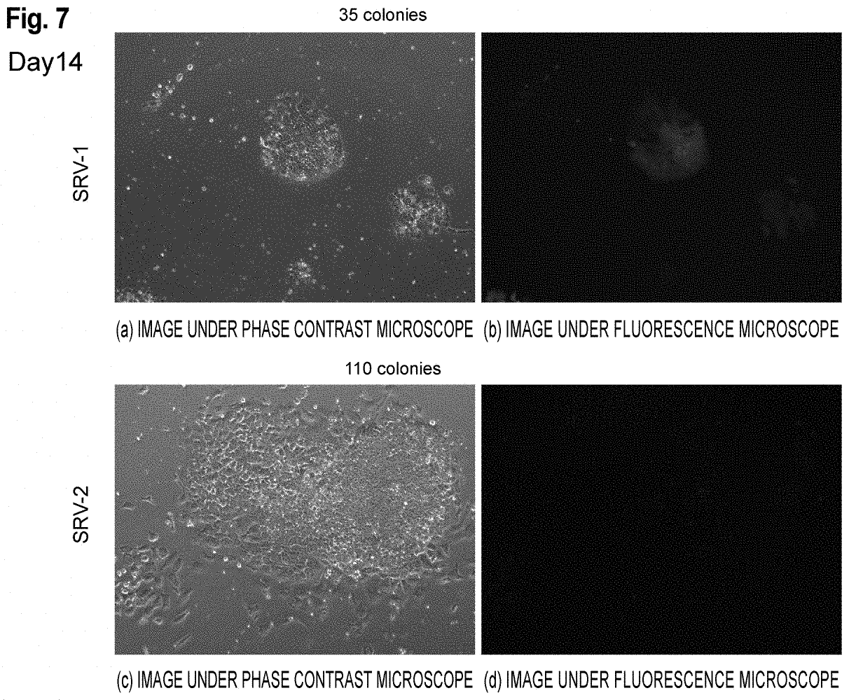

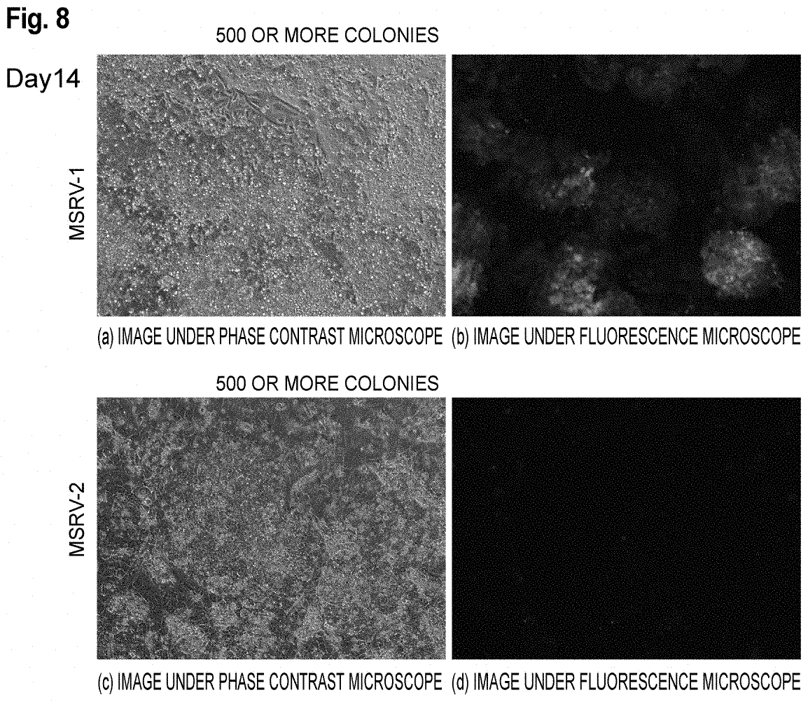

FIG. 7 and FIG. 8 show images of the cells on the 14th day. The cells into which SRV-1 was introduced formed 35 colonies, and the cells into which SRV-2 was introduced formed 110 colonies. The cells into which MSRV-1 was introduced formed 500 or more colonies, and the cells into which MSRV-2 was introduced formed 500 or more colonies. Example 2 shows that the chimeric virus genomic RNA disappeared from the cells faster than the non-chimeric virus genomic RNA. In addition, Example 2 shows that the chimeric virus had a high stem cell induction efficiency.

### Example 3

An αMEM medium to which FBS having a final concentration of 20% and penicillin/streptomycin having a final concentration of 1% were added was prepared as a medium for OP9 cells.

A medium for OP9 cells to which vitamin C having a final concentration of 100 mmol/L, interleukin-7 (IL-7) having a final concentration of 10 μg/mL, FLT3 ligand having a final concentration of 10 μg/mL, and a stem cell factor (SCF) having a final concentration of 10 μg/mL were added was prepared as a medium for inducing differentiation.

The colonies of iPS cells induced in Example 2 were detached from the culture container with 0.25% of trypsin and 1 mg/mL of collagenase IV, and divided into a plurality of cell masses by pipetting. A culture container in which OP9 cells and OP9/DLL1 cells were cultured as feeder cells was prepared. The OP9 cells and OP9/DLL1 cells were cultured in the medium for OP9 cells. The plurality of cell masses composed of iPS cells were seeded on the feeder cells, and the cells were cultured using the medium for OP9 cells to which vitamin C was added for 14 days.

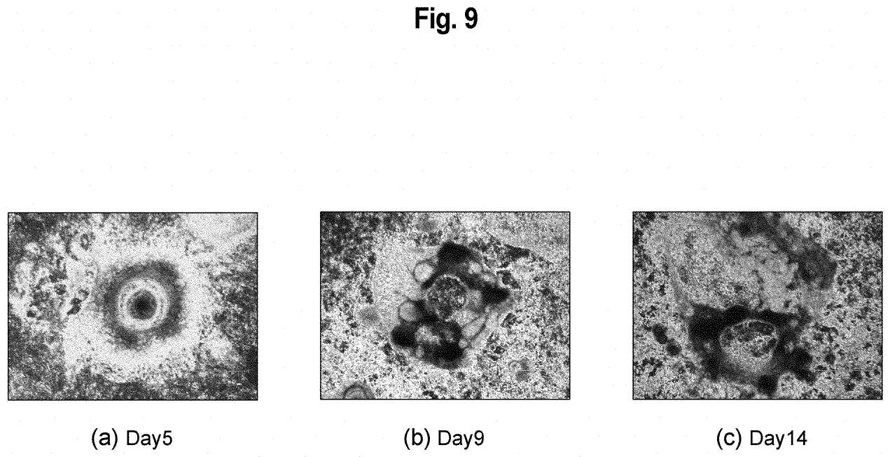

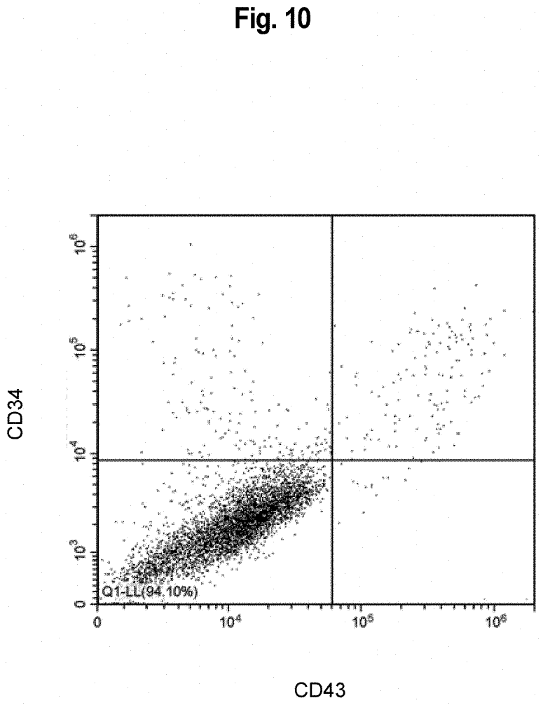

The iPS cells were detached from the culture container, seeded in a culture container in which the OP9 cells and OP9/DLL1 cells were newly cultured, and the cells were cultured using the medium for inducing differentiation. FIG. 9 shows images of the cells on the 5^{th} day, 9^{th} day and 14^{th} day after the iPS cells were seeded on the OP9/DLL1 cells. A progress of the iPS cells differentiated into blood progenitor cells was observed. In addition, FIG. 10 shows the results obtained by analyzing cells on the 14^{th} day with a flow cytometry. In the cells, it was confirmed that blood cell markers CD34 and CD43 were positive. Then, on the 16^{th} day, 23^{th} day, and 30^{th} day, only floating cells were collected, and seeded and passaged in a culture container in which the OP9 cells and OP9/DLL1 cells were newly cultured.

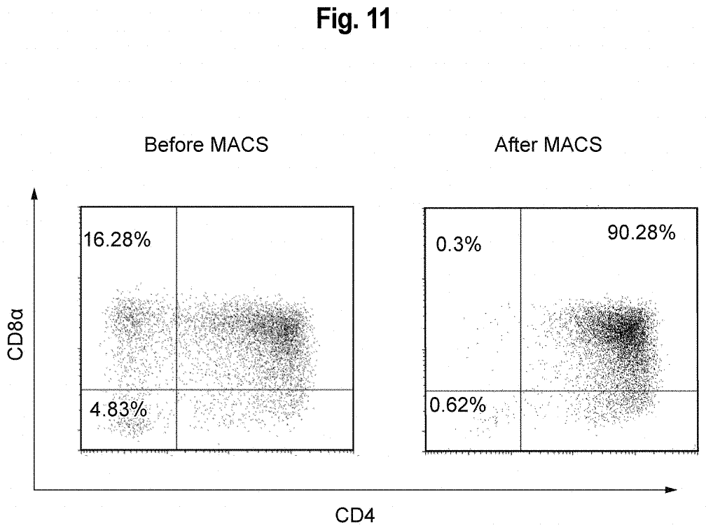

On the 37^{th} day after the iPS cells were seeded on the feeder cells, as shown in FIG. 11, the induced blood cells were sorted for CD4-positive and CD8α-positive cells using anti-CD4 microbeads according to a magnetic-activated cell sorting (MACS) method.

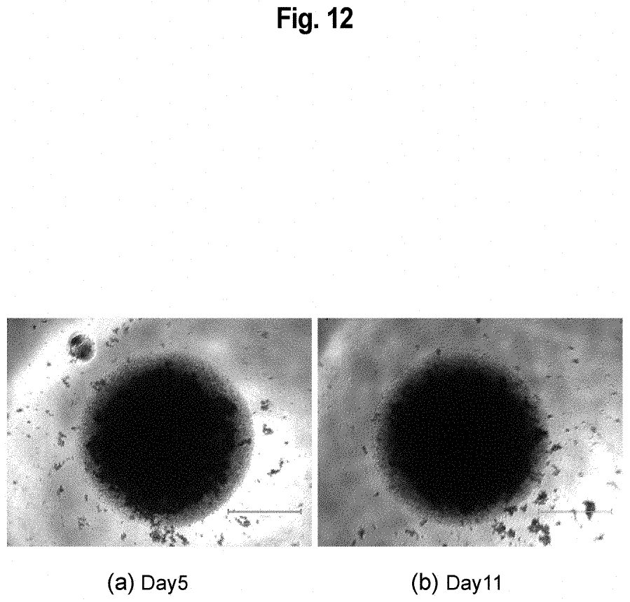

The cells sorted by MACS were cultured using the medium for inducing differentiation, and anti-CD3 antibodies (with a final concentration of 50 ng/mL), anti-CD28 antibodies (with a final concentration of 2 ng/mL), and interleukin-2 (IL-2, final concentration of 200 U/mL) were added to the medium. FIG. 12 shows images of the cells on the 5^{th} day and the cells on the 11^{th} day after sorting by MACS.

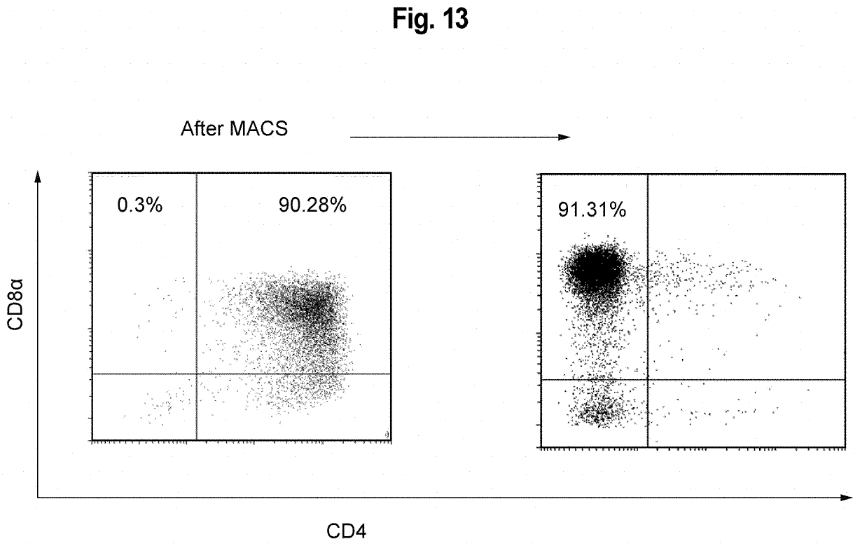

As shown in FIG. 13, the cells on the 10^{th} day after sorting by MACS were analyzed with a flow cytometer and they were CD4-negative and CD8α-positive. Example 3 shows that CD8a single positive cells which can differentiate into cytotoxic T cells could be differentiated from iPS cells.

### Example 4

A blood cell medium (RPMI, ThermoFisher) containing IL-2 having a final concentration of 20 ng/mL (Peprotech), FBS having a final concentration of 10% (cytiva), and zoledronic acid having a final concentration of 5 mmol/L was prepared. In addition, MSRV-2 (TOKIWA Bio Inc.) was prepared.

10 mL of the blood cell medium was put into a 15 mL tube, and human γδT cells were additionally added. Next, 200 g of the γδT cells was centrifuged for 5 minutes. After centrifugation, the γδT cells were collected, the number of the γδT cells was counted, and 5×10⁵ γδT cells were isolated. 10 mL of the blood cell medium was put into a 15 mL tube and 5×10⁵ γδT cells were additionally added. Next, 200 g of the γδT cells was centrifuged for 5 minutes. After centrifugation, the blood cell medium was removed from the tube, and 1 mL of the new blood cell medium was put into the tube. The γδT cells were suspended in the blood cell medium, and the γδT cells were seeded in a non-laminin-coated 26-well dish. Then, the γδT cells were cultured in an incubator at 37° C. for 3 days. IL-2 was added to the medium once a day.

The γδT cells were collected from the well-dish, and 200 g of the γδT cells was centrifuged for 5 minutes. After centrifugation, the blood cell medium was removed from the tube, and 2 mL of the new blood cell medium was put into the tube. The γδT cells were suspended in the blood cell medium, and the γδT cells were seeded in a non-laminin-coated 26-well dish. Then, the γδT cells were cultured in an incubator at 37° C. for 3 days.

1×10⁵ γδT cells were isolated, and the γδT cells were seeded in a 96-well dish containing the blood cell medium. In addition, MSRV-2 was added to the blood cell medium of each dish. Thereby, KLF4, OCT3/4, SOX2, and c-MYC were introduced into the γδT cells. The multiplicity of infection (MOI) was adjusted from 3 to 30.

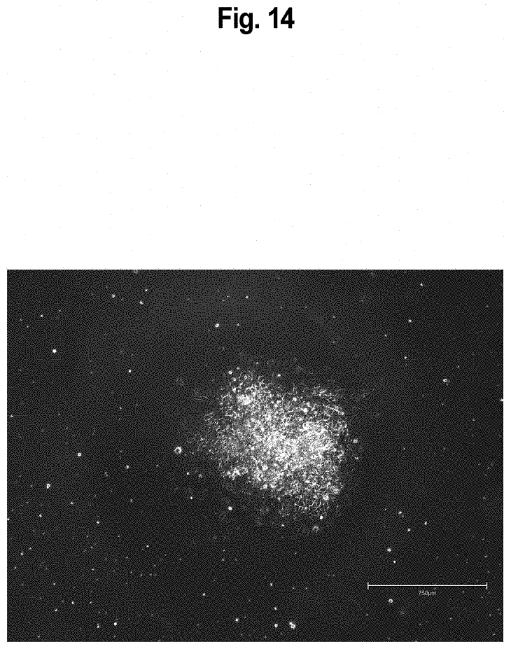

On the 3^{rd} day after the virus was added to the blood cell medium, the blood cell medium was removed from the dish, the cells were washed with a stem cell medium (StemFit, registered trademark, Ajinomoto), and 1 mL of the stem cell medium was then added to the dish. Then, the medium was replaced once every two days, and passaging was performed whenever colonies were formed. As shown in FIG. 14, iPS-cell-like cells were observed. Example 4 shows that stem cells were induced from γδT cells using a chimeric virus.

What is claimed is:

1. A method for producing stem cells, comprising:
preparing somatic cells;
preparing a chimeric virus including a virus-derived genomic RNA harboring an inducer RNA that induces somatic cells into stem cells and a virus-derived envelope surrounding the genomic RNA, wherein the genomic RNA and the envelope are derived from different viruses; and
introducing the inducer RNA into the somatic cells using the chimeric virus.

2. The method for producing stem cells according to claim 1,
wherein the genomic RNA is derived from paramyxovirus.

3. The method for producing stem cells according to claim 1,
wherein the genomic RNA is derived from a Sendai virus.

4. The method for producing stem cells according to claim 1,
wherein the genomic RNA includes Sendai virus genes in which all functions of M genes, F genes and HN genes among Sendai virus genes including NP genes, P genes/C genes, M genes, F genes, HN genes, and L genes are defected and having a mutation in the L gene that allows permanent gene expression.

5. The method for producing stem cells according to claim 1,
wherein the envelope is derived from a measles virus.

6. The method for producing stem cells according to claim 1,
wherein the somatic cells are blood cells.

7. The method for producing stem cells according to claim 6,
wherein the blood cells are blood cells that do not contain T cells.

8. The method for producing stem cells according to claim 6,
wherein the blood cells are T cells.

9. The method for producing stem cells according to claim 1, further comprising
applying a phosphate to the somatic cells.

10. The method for producing stem cells according to claim 9,
wherein the phosphate is an intermediate or final product of a non-mevalonate pathway.

11. The method for producing stem cells according to claim 9,
wherein the phosphate is (E)-4-hydroxy-3-methyl-2-butenyl diphosphate.

12. The method for producing stem cells according to claim 1, further comprising
applying a bisphosphonate to the somatic cells.

13. The method for producing stem cells according to claim 1, further comprising
applying an interleukin to the somatic cells.

14. The method for producing stem cells according to claim 1,
wherein, when cells into which the inducer RNA is introduced are passaged, at least some of the collected and mixed cells are seeded.

15. The method for producing stem cells according to claim 1,
wherein, when cells into which the inducer RNA is introduced are passaged, the cells are seeded at a low concentration.

16. A method for producing somatic cells, comprising:
preparing the stem cells produced according to the method for producing stem cells according to claim 1; and
inducing somatic cells from the stem cells.

17. The method for producing somatic cells according to claim 16,
wherein the somatic cells are blood cells.

18. The method for producing somatic cells according to claim 17,
wherein the blood cells are T cells.

19. The method for producing somatic cells according to claim 16,
wherein, when the inducer RNA is introduced into the stem cells, cell masses of the stem cells are seeded on feeder cells.

20. The method for producing somatic cells according to claim 19,
wherein the feeder cells are stromal cells.
